(19) United States
(12) Patent Application Publication
(10) Pub. No.: US 2018/0035952 A1
(43) Pub. Date: Feb. 8, 2018

(54) Concussive Impact Sensing Mouthguard

(71) Applicant: Keenan Matthew Fraylick, New Madison, OH (US)

(72) Inventor: Keenan Matthew Fraylick, New Madison, OH (US)

(21) Appl. No.: 15/226,900

(22) Filed: Aug. 2, 2016

(51) Int. Cl. A61B 5/00; A61B 5/11; A63B 71/08

(52) U.S. Cl. CPC A61B 5/7275; A63B 71/085; A61B 5/4064; A61B 5/11; A61B 5/6802; A61B 5/0022; A61B 2562/0219; A61B 2560/0214; A61B 2503/10

(57) ABSTRACT

A mouthguard capable of sensing high concussive risk collisions via the utilization of linear and rotational acceleration calculations. With pre-programmed biometric data of the user, the mouthguard will assess individual impacts sustained by the subject in accordance to a determined concussion threshold. Intended to sense possible neurological injuries, the mouthguard enables wounded users to receive needed protocol and treatment before further damage is sustained. The mouthguard will be capable of calculating, logging, and distributing impact force data to onlooking third parties, like coaches, parents, and medical personnel.

### CROSS-REFERENCE TO RELATED APPLICATIONS

(NOT APPLICABLE)

### FEDERALLY SPONSORED RESEARCH OR DEVELOPMENT

(NOT APPLICABLE)

### REFERENCE TO A SEQUENCE LISTING, A TABLE, OR A COMPUTER PROGRAM LISTING COMPACT DISC APPENDIX

(NOT APPLICABLE)

### BACKGROUND OF THE INVENTION

The present invention primarily pertains to the realm of devices or equipment with the intention of injury prevention. Specifically, the product aims to combat cerebral contusion and monitor concussive forces experienced by sport participants.

In detail, the innovation at hand correlates with a system lined with linear and angular acceleration sensors that calculate force associated with a particular impact undergone by its user. The system will also be capable of recording, storing, and transferring this collision data via Bluetooth application to onlooking medical or coaching staffs. Intended to be worn during times of physical activity, the utilization of this device through a mouthpiece allows versatile usage in a broad array of concussive-endangered sports and activities, like football, hockey, lacrosse, and soccer.

A concussion is a type of traumatic brain injury that transpires when the head experiences sudden linear or rotational motion changes; this causes the cerebrum to shake and crash into the skull, resulting in mild to severe bruising or swelling of the brain. Furthermore, delicate neural pathways of the cerebrum may become damaged and prompt neurological disturbances. According to the Center for Disease Control and Prevention, an estimated 3.8 million sports and recreation-related concussions occur annually. Unfortunately, up to 47% of these neurological injuries are undetected by provisional medical staffs or ignored by their victims. In the long term, 39% of all concussions lead to permanent neurological disabilities, like: Parkinson's Disease, Post Traumatic Stress Disorder, Bipolar Disorders, or Schizophrenia. In the realm of high school and collegiate-level athletics, the top four concussion-entrenched sports are football, ice hockey, lacrosse, and soccer, respectively. Varsity football at the high school tier accounts for nearly 63% of all concussions sustained by athletes ages ten through eighteen, while hockey deems nearly 11% of the mentioned statistic.

Although many leagues, organizations, and teams have improved the concussion protocol for their athletes, monitoring and detecting cerebral injuries can be problematic; the symptoms that follow a neural contusion, like dizziness, nausea, headache, or weakened coordination, can be mild without any indication of a serious neurological injury. When the athlete or medical provider deems these indications insignificant, it puts the athlete at risk of Second Impact Syndrome (SIS) when they return to physical activity. SIS arises when a concussion-affected athlete receives an impact to the head before the symptoms of the earlier one has subsided; this causes rapid and catastrophic cerebral edema, or swelling of the brain. SIS can lead to permanent neural disabilitation and may prove fatal in specific circumstances.

Due to the popularity of collision-based activities, a majority of sustained concussions are acquired by the youth. In 2015, nearly 75% of all cerebral contusions transpired with athletes ages ten to eighteen; for the typical high school male, 30% will endure a concussion before graduation. These statistics relentlessly expand as new studies help the scientific community better understand concussions, and as advanced medical equipment is fabricated to detect the neural bruising. In the National Football League alone, concussions have risen 58% between the 2014 and 2015 seasons. For these reasons, fans, parents, and athletes alike have voiced support for better protection and prevention from the cerebral injury. In response, the past decade has witnessed countless private and governmental funds that have been spent on concussion research, awareness, and prevention programs. Although these rudimentary actions have informed the public about the dangers of concussive-impact activities, data of annual concussions eerily and exponentially enlarges with each passing annum.

The system of the present invention correlates the concept behind linear and angular acceleration with traumatic brain injuries; when a component's acceleration drastically changes, so does the figure of force. During an impact, the changes in the head's velocity in correlation to time will output accelerations and force measurements of the collision's magnitude. Additionally, due to the pivot behavior of the cervical vertebrae, rotational forces can play a drastic role in collision-based concussions. Using three-axis accelerometers and gyroscopes, both the linear and rotational factors can be accounted in impact data measurements.

The process of assimilating impact force and accelerometer sensors in conventional sports equipment is not contemporary; prior arts have included numerous helmet-mounted monitoring systems that can calculate collision data and compute probabilities of neural contusion for its users. Unfortunately, these innovations do not integrate well with all sports including those which lack any major injury prevention equipment, like soccer and rugby. Additionally, many sports equipment corporations have worked to developed more advanced helmets and head protection. Due to the nature of concussive impacts, these head garments can not efficiently prevent cerebral bruising, only slow the duration of the impact to the user's cranium. Incorporating the monitoring system within the mouthguard component enables versatility to all contact sports, as it does not interfere with the activities' rules, regulations, nor their environments. Furthermore, the present invention enables a single, simple device instead of a complicated, multi-part system which may be cumbersome to employ or utilize on the user.

With regards to biometric data (age, weight, sex, height, etc.), the present invention will enable a low-cost force monitoring system with the capability of calculating linear and angular accelerations of the head and computing a corresponding concussive risk factor to particular impacts.

The present innovation and the actions it performs will be described in greater detail in the description below.

### BRIEF SUMMARY OF THE INVENTION

The present invention is a mouthguard intended for athletic and recreational usage that is capable of calculating, logging, and distributing collision data to appropriate onlookers, like: coaches, medical staffs, and parents. Overcoming the disadvantages of prior arts, the device's versatility permits the product's utilization in several sports. Additionally, the system's simple and elementary setup enables a cost-efficient and accessible product that can easily be incorporated into the user's activity.

By configuring the three-axis accelerometer, gyroscope, and data logger between the mouthguard's outer shell and teeth mold, it will enable safe user operation without fear or risk of interference from electronic entities. Intended to be worn during times of athletic or recreational activity, the three mentioned instruments are capable of calculating and logging impact force data when the user's head experiences a drastic change in linear or rotational acceleration that can provoke traumatic head trauma. As a power source, the present invention will also contain a battery, ideally embedded in the electronic and data board containing the sensors and data logger. To conserve energy, an on/off switch may be implemented on the mouthguard, in which the system's data logger or processing unit maintains its flash memory and past collision data even when the present invention ceases power. In the case of a power failure, a backup battery may be employed to resume operation until the main power source can be recharged. Furthermore, the system will include a low-energy Bluetooth device entitled to broadcast and transmit real-time collision data over short distances to a software (or a mobile device incorporating said software) that can determine the risk factor with each athletic collision. In regards to biometric data (height, weight, sex, etc.), the mentioned software will establish a threshold which indicates if an impact is potentially concussive and if the user should be tested and examined for neurological disturbances. For example, an average, high school male with a height of 5′8″ and weight of 150 lbs. would undergo concussion protocol if the force exceeded their concussive threshold of 80 g-force. Although concussions can potentially occur at any impact force, the use of a pre-established thresholds enable much needed, but not abundant examinations of concussion-endangered athletes and users.

In regards to the present invention's sensors, the accelerometer and gyroscope will both be located parallel to the location of the user's second molars in the rear of the mouth; if positioned in this site, the sensors would have a stronger correlation to the subject's neural center of gravity. Furthermore, since these teeth are directly affixed to the skull, the positioning of the sensors will permit more precise impact force data when in operation.

The present invention will utilize an onboard central processing unit (CPU) and memory storage unit, perhaps a common random-access memory (RAM) or flash memory. Memory storage may be temporary, in which impact data refreshes from the unit every 48 hours or after every recharge of the system's battery. The CPU and its hard-coded instructions will read and transmit each impact force measurement to both the memory storage unit and the low-powered Bluetooth entity. It is the role of the independent software to read this collision data and deem the concussion risk factor involved in each collision communicated via the mouthguard and CPU.

### BRIEF DESCRIPTION OF THE SEVERAL VIEWS OF THE DRAWINGS

A more complete understanding of the present invention and its design can be accomplished via observation of the images provided below and its descriptions thereof:

FIG. 1 depicts a top view of the electronic and data board of the present invention.

FIG. 2 depicts an iso view of the base layer of the present invention.

FIG. 3 depicts an iso view of the teeth mold coating of the present invention.

FIG. 4 depicts an exploded view of the layers of the present invention.

### DETAILED DESCRIPTION OF THE INVENTION

A more complete understanding of the present invention can be achieved through the descriptions of the various embodiments employed by the system.

The present invention incorporates a mouthguard, a common and universal injury prevention device utilized by all realms of athletes, to calculate, log, and distribute impact force data to a software (or personnel with access to the said software) that is able to compute concussive risk factors of specific hits. Using embodiments that are capable of calculating both linear and angular accelerations, the central processing unit of the present invention can manipulate and manage the given information in accordance to traumatic head trauma probabilities. The system may integrate the user's biometric data in reference to the stated risk factor, or utilize a specific force threshold that will compel the standard concussion protocol to be conducted on the affected user and persona. The present invention will also employ both a memory storage unit, like a RAM, a dual-set battery to power the electronic and data board, and a low-energy Bluetooth to distribute the collision data to specified third parties.

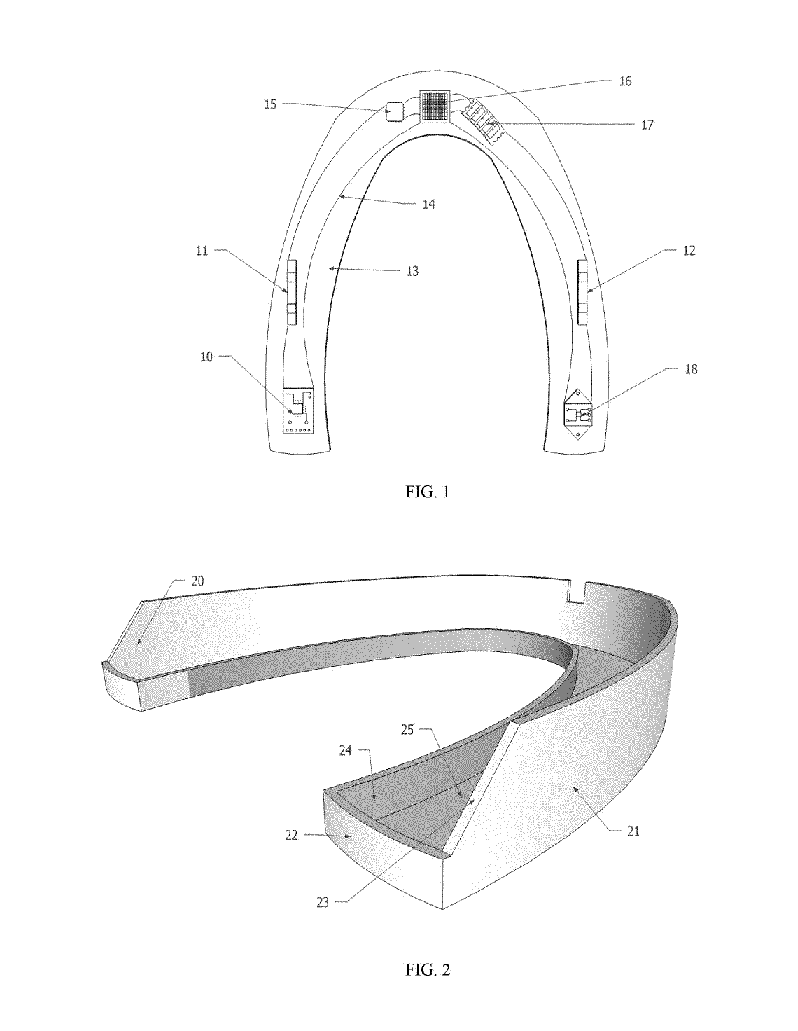

The present invention is divided into three sections: the base layer, the electronic and data board, and the teeth mold coating. In FIG. 1, the diagram depicts the most vital section of the three: the electronic and data board. Situated between the base layer and the teeth mold coating, the said board will accommodate a majority of the present invention's embodiments and complex entities. Embodiment 10 shall be located on the left section of the board, ideally located near the bottom subsection parallel to the user's molar teeth; embodiment 10 will be a gyroscope, with the capability of measuring the user's rotational acceleration when the said subject is involved in a collision. This body will work together with embodiment 18, a three-axis accelerometer, in the overall computation of the impact force data. Body 18 will instead calculate the linear acceleration involved in a collision; ideally, this embodiment will be located in the lower right subsection of the said electronic and data board. Both embodiment 10 and 18 shall be micro-entities, not exceeding the dimensions of 0.5″, 0.5″, 0.05″. Both of these devices will be connected to embodiment 16 via the utilization of body 14. Embodiment 16 will be a central processing unit (CPU) with a cache storage unit of no less than 1 MB; furthermore, the entity shall function at a speed of no less than 1 GHz. Embodiment 16 should resemble the size of the accelerometer and gyroscope. All parts of the board may be connected via embodiment 14, a simple, metallic, single threaded wiring complex circumventing the apparatus. Alternatively, instead of the wiring complex, embodiment 13 may be a flexible circuit board custom-fitted to the specific mouthguard.

Embodiments 11 and 12 are both batteries intended to power the apparatus; one may be the primary power source while the other functions as a backup battery in the case of energy failure. Both may contain charging ports and an on/off switch to prolong the present invention's lifespan and functioning time. Ideally, both entities should not exceed the dimensions of 0.25″, 0.5″, 0.05″. Embodiment 17 will be connected to body 16, the CPU, and will act as a memory storage unit to log and accumulate collision data for a period up to 48 hours, or until the present invention's next recharge section; specifically, this entity may be a RAM implement with a similar size comparison to the two batteries of the system. Lastly, embodiment 15 will be the low-energy Bluetooth entity of the apparatus. Similar to the size of embodiments 10 and 18, the body shall run at a speed no less than 16 MHz and contain a flash memory of no less than 300 KB. Additionally, the entity shall distribute collision data to a third party software that is capable of computing concussive risk factors involved in each impact.

FIG. 2 depicts the primary, base layer of the mouthguard. Embodiment 20, 21, 22, 23, 24, and 25 shall all be constructed out of a hard, hydrophobic polyethylene-polyvinylacetate copolymer that will resist water deterioration, while prolonging the lifespan of the present invention. Embodiment 20 and 21, the inside and outside of the primary fitted plate respectively, will be molded at the angles involved in the user's teeth curvature. Body 20 may be glazed with a softer plastic molding to reduce gum irritation when in activity. Embodiment 22, the rear incline and section of the mouthguard, shall be of a height of no less than 0.5″ to properly fit the subject's teeth and prevent slipping of the present invention from its intended position. Embodiment 24 shall be of the same height as body 22 and may contained hinges or ledges to lock the electronic board and teeth mold coating into their desirable locations within the base layer. Embodiment 25, or the bottom section of the base layer, shall be at a height of no less than 0.125″. Lastly, body 23 should connect embodiments 22 and 21 with an angled incline of no less than 45 degrees, transgressing from 0.5″ to 1″.

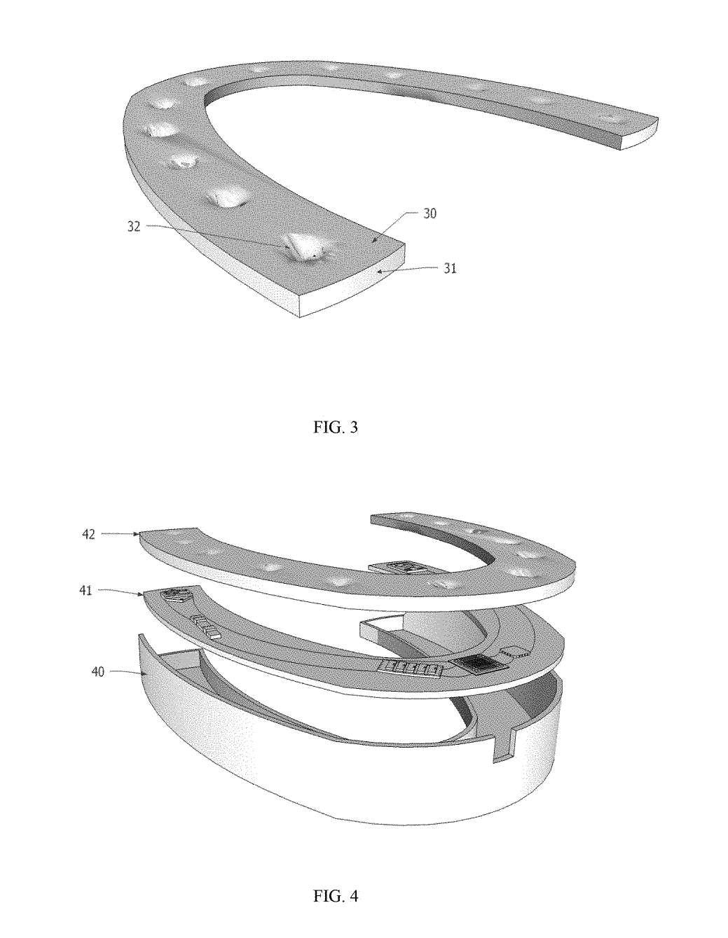

FIG. 3 depicts the teeth mold coating section of the present invention. Embodiment 30, or the material of the stated coating, shall be a soft rubber material with a melting point of no less than 150 degrees Celsius. Between the temperatures of 90-120 degrees Celsius, this soft rubber should be able to be freely manipulated and molded to the dimensions of the user's specific teeth shapes and sizes. A sample image of this teeth mold is found in embodiment 32, which will vary in size and dimension based on the type of tooth and the subject's biometric profile. Lastly, embodiment 31 shall be at a height of no less than 0.25″ to enable a complete fitting of the entire teething section.

FIG. 4 depicts an exploded view of all the layers of the present invention. Body 40, the base layer, shall be located at the bottom with the electronic and data board (Body 41) and teeth mold coating (Body 42) following. All the given layers may be affixed via an adhesive, water resistant glue or hinges located on the inside section of the mouthguard's base layer.

I claim:

1. A mouthguard that calculates, logs, and distributes impact force data and the magnitude thereof in reference to a concussive risk factor of particular collisions.

2. The mouthguard of claim 1 wherein said at least three distinctive layers: the base layer, the electronic and data board, and the teeth mold coating.

3. The electronic and data board of claim 2 wherein said fabricated with a wiring complex between entities, or a flexible circuit board between the base layer and teeth mold coating.

4. The mouthguard of claim 1 wherein said at least one gyroscope and one three-axis accelerometer, capable of computing the rotational and linear accelerations respectively.

5. The mouthguard of claim 1 wherein said at least one central processing unit to categorize and manage the impact force data distributed thereof.

6. The central processing unit of claim 5 wherein said at least 1 MB of cache memory and a functioning speed of no less than 1 GHz.

7. The central processing unit of claim 5 wherein said input data of user specific biometric data for computation of the stated concussive risk factor.

8. The mouthguard of claim 1 wherein said at least one memory storage unit, particularly a random-access memory unit, with the ability to accumulate and log up to 48 hours worth of collision data.

9. The mouthguard of claim 1 wherein said at least one low-energy Bluetooth unit to distribute collision data to an integrated software over short distances.

10. The electronic and data board of claim 2 wherein said at least two power sources, particularly batteries, compatible with the power-dependent entities of the said board.

11. The mouthguard of claim 1 wherein said a base layer constructed of a hard, hydrophobic polyethylene-polyvinylacetate copolymer.

12. The base layer of claim 11 wherein said hinges or slots to thereof lock the positioning of the other two stated layers.

13. The mouthguard of claim 1 wherein said an upper teeth mold coating constructed of a soft, durable, and malleable rubber material.

14. The batteries of claim 10 wherein said an on/off switch and charging port for operation.
